(19) United States
(12) Patent Application Publication
(10) Pub. No.: US 2015/0073148 A1
(43) Pub. Date: Mar. 12, 2015

(54) PROCESS FOR THE PREPARATION OF CRYSTALLINE VILAZODONE HYDROCHLORIDE

(71) Applicant: RANBAXY LABORATORIES LIMITED, New Delhi, Delhi (IN)

(72) Inventors: Prasenjit Prafulla Das, Nagpur (IN); Bindu Srivastava, North West Delhi (IN); Nitin Maheshwari, New Delhi (IN); Hashim Nizar Poovanathil Nagoor Meeran, Pathanamthitta (IN); Mohan Prasad, Gurgaon (IN); Sudershan Kumar Arora, Gurgaon (IN)

(21) Appl. No.: 14/394,542

(22) PCT Filed: Apr. 16, 2013
(86) PCT No.: PCT/IB2013/053024

§ 371 Date: Oct. 15, 2014

(30) Foreign Application Priority Data
Apr. 16, 2012 (IN) 1173/DEL/2012

(51) Int. Cl. C07D 405/12

(52) U.S. Cl. CPC C07D 405/12
USPC 544/373

(57) ABSTRACT

The present invention relates to a process for the preparation of crystalline vilazodone hydrochloride.

### FIELD OF THE INVENTION

The present invention relates to a process for the preparation of crystalline vilazodone hydrochloride.

### BACKGROUND OF THE INVENTION

Vilazodone is chemically described as 5-{4-[4-(5-cyano-1H-indol-3-yl)butyl]piperazin-1-yl}-1-benzofuran-2-carboxamide of Formula I.

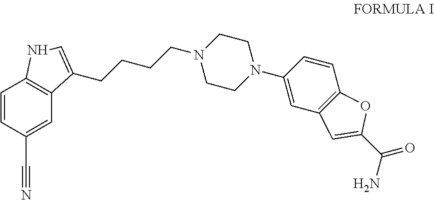 [SMILES: N#Cc1ccc2[nH]cc(CCCCN3CCN(c4ccc5oc(C(N)=O)cc5c4)CC3)c2c1]

Vilazodone is indicated for the treatment of major depressive disorder (MDD).

Processes for the preparation of vilazodone free base or its hydrochloride are described in U.S. Pat. Nos. 5,532,241 and 7,834,020; and European Patent Nos. EP 0 648 767 and EP 1 397 357.

### SUMMARY OF THE INVENTION

The present invention relates to a process for the preparation of crystalline vilazodone hydrochloride.

### BRIEF DESCRIPTION OF THE DRAWINGS

FIG. 1 depicts the X-ray powder diffraction pattern (XRPD) of the crystalline vilazodone hydrochloride obtained according to Example 1.

FIG. 1A provides the table of values for the XRPD pattern depicted in FIG. 1.

FIG. 2 depicts the X-ray powder diffraction pattern (XRPD) of the crystalline vilazodone hydrochloride obtained according to Example 2.

FIG. 2A provides the table of values for the XRPD pattern depicted in FIG. 2.

FIG. 3 depicts the X-ray powder diffraction pattern (XRPD) of the crystalline vilazodone hydrochloride obtained according to Example 3.

FIG. 3A provides the table of values for the XRPD pattern depicted in FIG. 3.

FIG. 4 depicts the X-ray powder diffraction pattern (XRPD) of the crystalline vilazodone hydrochloride obtained according to Example 4.

FIG. 4A provides the table of values for the XRPD pattern depicted in FIG. 4.

FIG. 5 depicts the X-ray powder diffraction pattern (XRPD) of the crystalline vilazodone hydrochloride obtained according to Example 5.

FIG. 5A provides the table of values for the XRPD pattern depicted in FIG. 5.

FIG. 6 depicts the X-ray powder diffraction pattern (XRPD) of the crystalline vilazodone hydrochloride obtained according to Example 6.

FIG. 6A provides the table of values for the XRPD pattern depicted in FIG. 6.

FIG. 7 depicts the X-ray powder diffraction pattern (XRPD) of the crystalline vilazodone hydrochloride obtained according to Example 7.

FIG. 7A provides the table of values for the XRPD pattern depicted in FIG. 7.

### DETAILED DESCRIPTION OF THE INVENTION

An aspect of the present invention provides a process for the preparation of crystalline vilazodone hydrochloride, which comprises:

a) treating vilazodone free base with hydrochloric acid in the presence of water and a solvent selected from the group consisting of alcohol, halogenated hydrocarbon, esters, or a mixture thereof.

b) isolating crystalline vilazodone hydrochloride from the reaction mixture thereof.

The vilazodone free base used as a starting material may be used in any solid form, and prepared according to the methods described in U.S. Pat. No. 5,532,241 or our co-pending Indian Patent Application No. IN 281/DEL/2012. Vilazodone free base used as a starting material may be used in the form of reaction mixture prepared in situ.

Vilazodone free base may be treated with hydrochloric acid in the presence of water and a solvent selected from the group consisting of alcohol, halogenated hydrocarbon, esters, or a mixture thereof. Suitable alcoholic solvents may include methanol, ethanol, 2-propanol, 1-propanol, or butanol. Preferable alcohol solvents may include 2-propanol, ethanol, or methanol. Suitable halogenated hydrocarbon solvents may include dichloromethane or chloroform. Preferable halogenated hydrocarbon solvents may include dichloromethane. Suitable ester solvents may include ethyl acetate, methyl acetate, or isopropyl acetate. Preferable ester solvents may include ethyl acetate.

Water may be added to the reaction mixture before or after the addition of hydrochloric acid.

The hydrochloric acid may be dilute or concentrated. The hydrochloric acid may be used in solution form or gaseous form. The solution of hydrochloric acid may be aqueous or in alcoholic solvent. The alcoholic solvent used for the preparation of hydrochloric acid solution may preferably be 2-propanol.

Treatment of vilazodone free base with hydrochloric acid may be carried out a temperature of about 10° C. to about 100° C., preferably at about 20° C. to about 85° C. Treatment of vilazodone free base with hydrochloric acid may be carried out for about 30 minutes to about 3 hours, preferably for about 1 hour to about 2 hours.

The vilazodone hydrochloride salt may be isolated by filtration, distillation, evaporation, centrifugation, decantation, drying, vacuum drying, or a combination thereof.

Crystalline vilazodone hydrochloride prepared by the present invention may be characterized using X-ray powder diffraction pattern (XRPD).

XRPD of the samples were determined by using Panalytical X'Pert Pro X-Ray Powder Diffractometer in the range 3-40 degree 2 theta, and under tube voltage and current of 45 Kv and 40 mA, respectively. Copper radiation of wavelength 1.54 angstrom and Xceletor detector was used.

In the following section, embodiments are described by way of examples to illustrate the process of invention. Several variants of these examples would be evident to persons ordinarily skilled in the art.

### Example 1

### Preparation of Vilazodone Hydrochloride

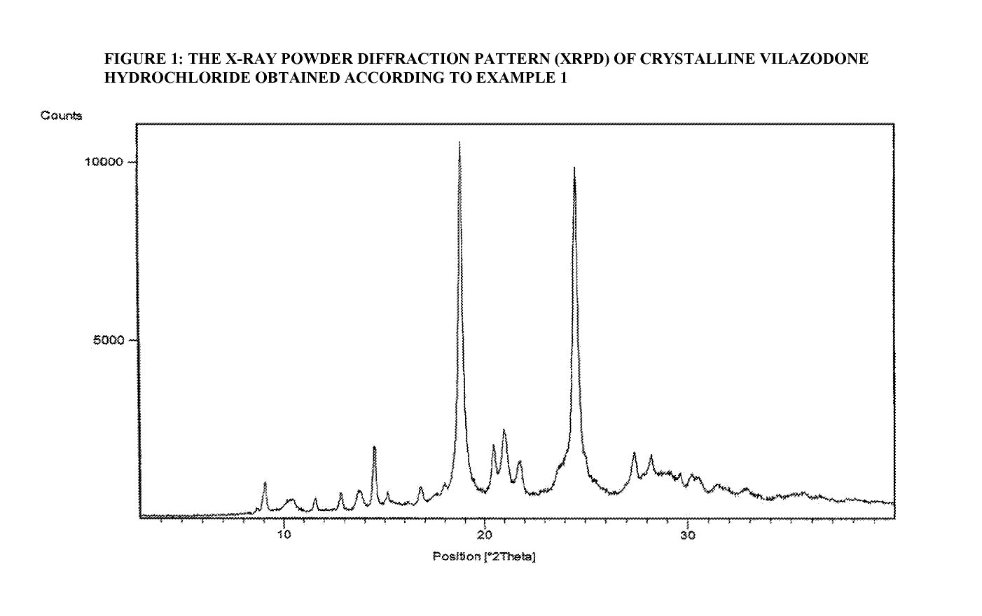

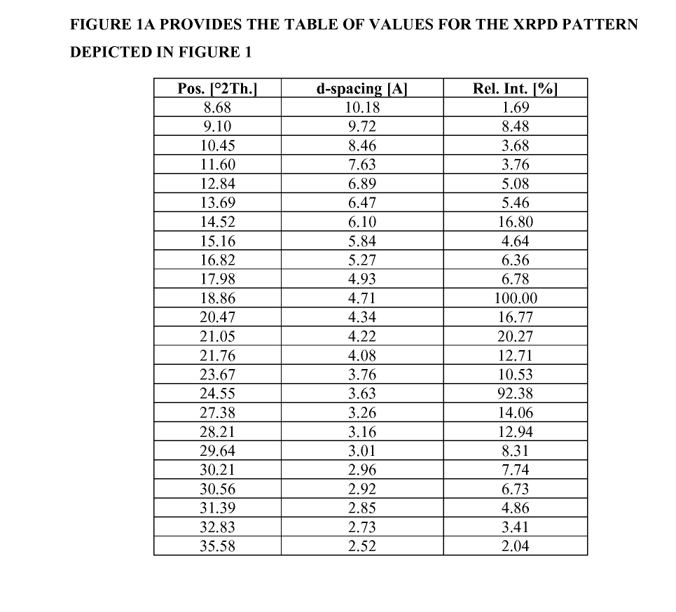

Vilazodone free base (20.0 g) was added to 2-propanol (860 mL). The reaction mixture was heated to 83° C., and water (40 mL) was added. Concentrated hydrochloric acid (4.7 g) was added to the reaction mixture at 80° C. to 83° C. and the mixture was stirred at 70° C. to 83° C. for 60 minutes. The solid obtained was filtered, washed with 2-propanol (40 mL), and dried under vacuum at 20° C. to 30° C. for 6 hours to obtain the title compound having XRPD data as shown in FIG. 1.

Yield: 13.0 g

### Example 2

### Preparation of Vilazodone Hydrochloride

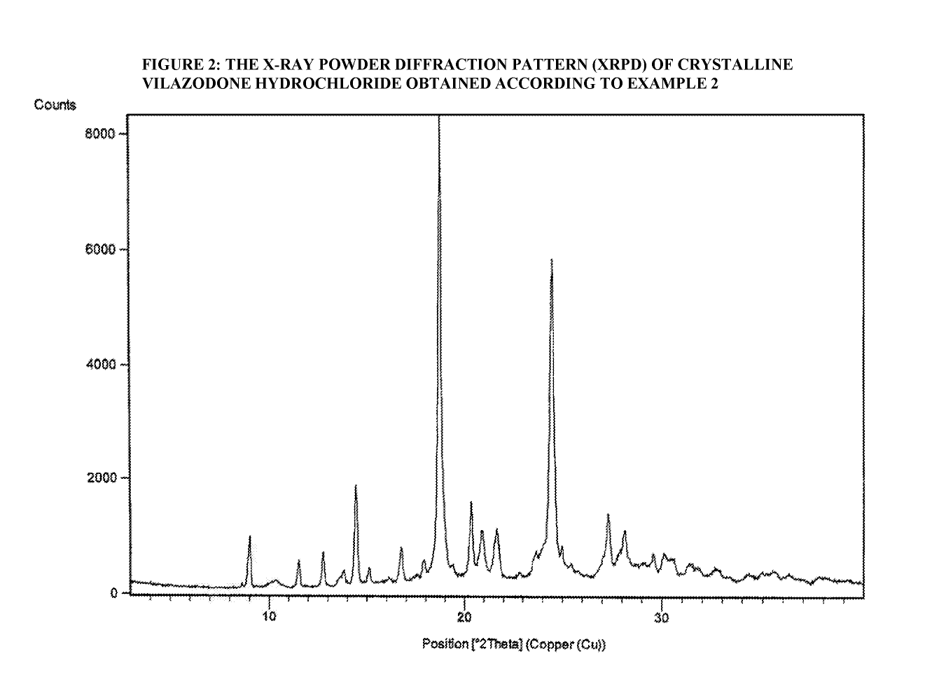

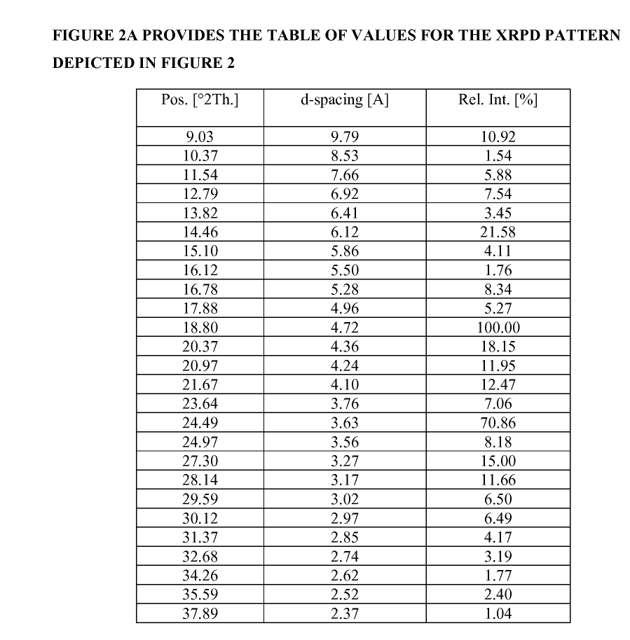

Vilazodone free base (20.0 g) was added to 2-propanol (860 mL). The reaction mixture was heated to 80° C. to 85° C., and water (40 mL) was added. Activated carbon (0.5 g) was added to the reaction mixture at 80° C. and the mixture was filtered. The reaction mixture was washed with 2-propanol (80 mL) at 75° C. to 80° C. 0.1N 2-propanolic hydrochloride (prepared by mixing concentrated hydrochloric acid (4.7 g) and 2-propanol (450 mL)) was added to the reaction mixture at 80° C. to 81° C. over 60 minutes. The reaction mixture was cooled to 60° C. over 60 minutes. The solid obtained was filtered, washed with 2-propanol (40 mL), and dried under vacuum at 20° C. to 30° C. for 16 hours to obtain the title compound having XRPD data as shown in FIG. 2.

Yield: 3.0 g

### Example 3

### Preparation of Vilazodone Hydrochloride

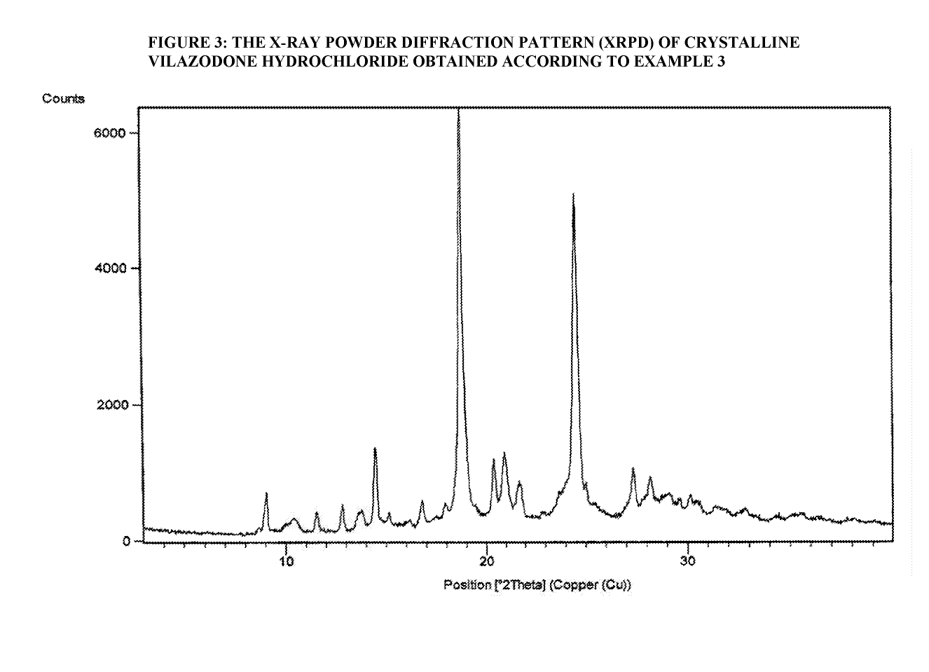

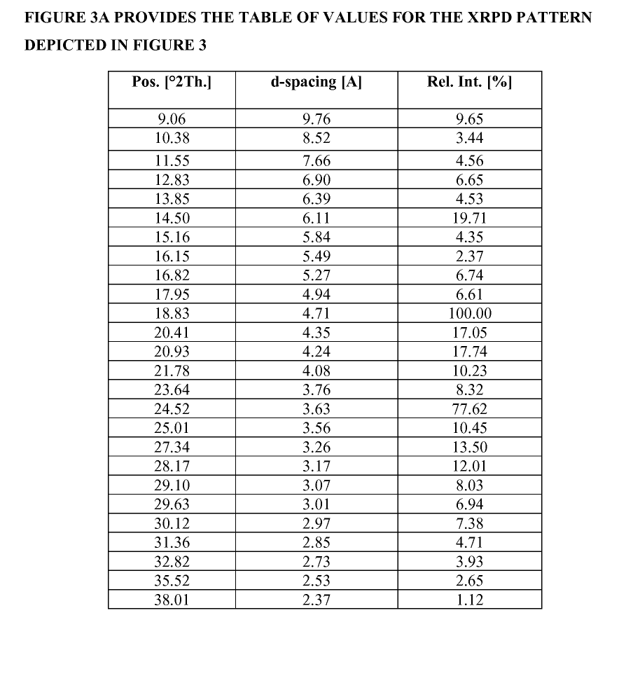

Vilazodone free base (4.0 g) was added to 2-propanol (172 mL). The reaction mixture was heated to 82° C., and water was added to the reaction mixture (8 mL). The reaction mixture was treated with activated carbon (0.2 g) at 80° C., filtered, and washed with 2-propanol (20 mL). 0.1N 2-propanolic hydrochloride (prepared by mixing concentrated hydrochloric acid (0.9 g), and 2-propanol (70 mL)) was added to the reaction mixture at 80° C. to 81° C. over 60 minutes. The reaction mixture was cooled to 60° C. and stirred at 60° C. for 30 minutes. The solid obtained was filtered, washed with 2-propanol (8 mL) at 60° C., and dried under vacuum at 50° C. to 55° C. for 16 hours to obtain the title compound having XRPD data as shown in FIG. 3.

Yield: 2.0 g

### Example 4

### Preparation of Vilazodone Hydrochloride

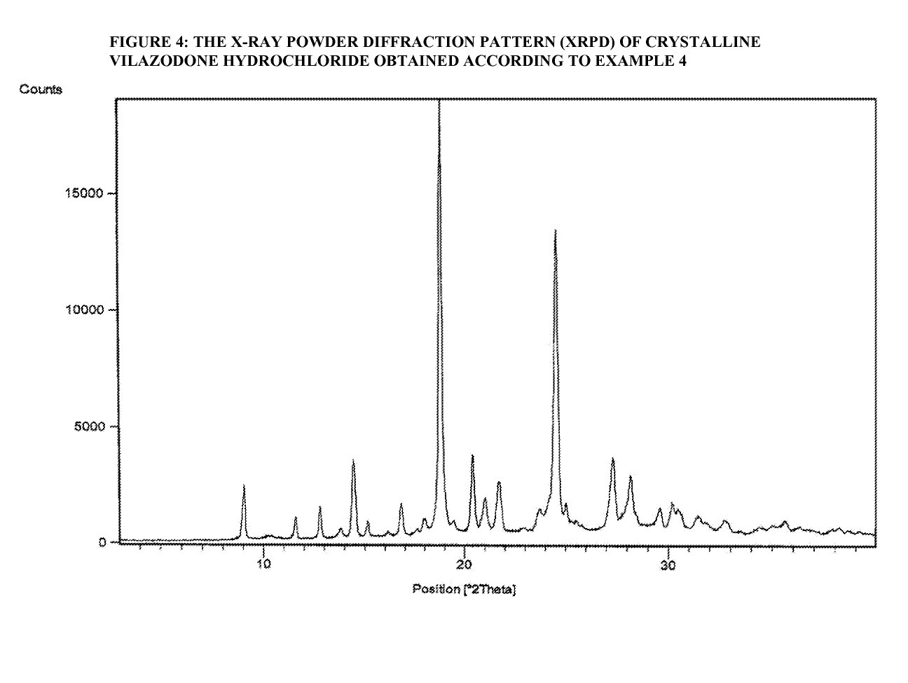

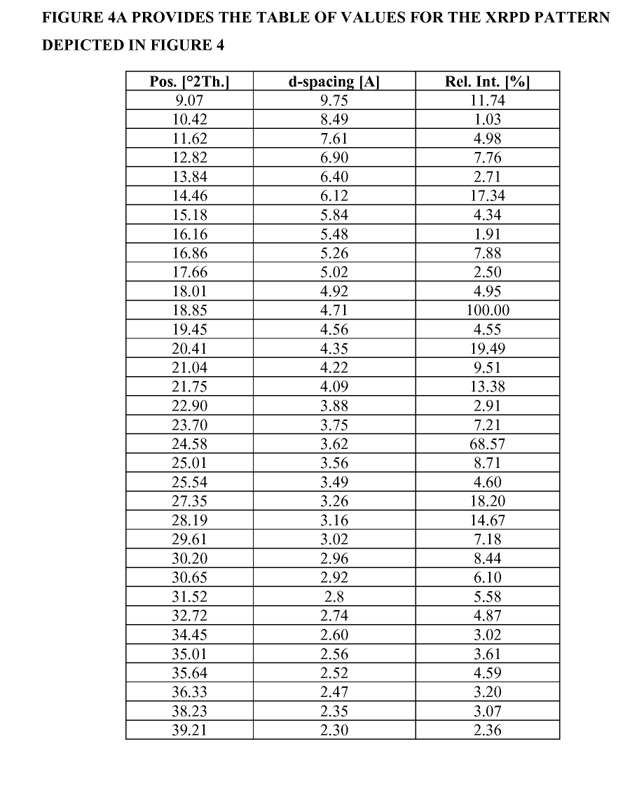

Vilazodone free base (60.0 g) was added to 2-propanol (2580 mL). The reaction mixture was heated to 80° C. to 83° C., and water (80 mL) was added. A solution of 0.1N 2-propanolic hydrochloride (1360 mL) was added to the reaction mixture at 65° C. to 70° C. over 60 minutes. The reaction mixture was cooled to 25° C. to 30° C. and stirred for 3 hours. The solid obtained was filleted, washed with diethyl ether (120 mL), and dried under vacuum at 20° C. to 30° C. for 16 hours to obtain the title compound having XRPD data as shown in FIG. 4.

Yield: 60.4 g

### Example 5

### Preparation of Vilazodone Hydrochloride

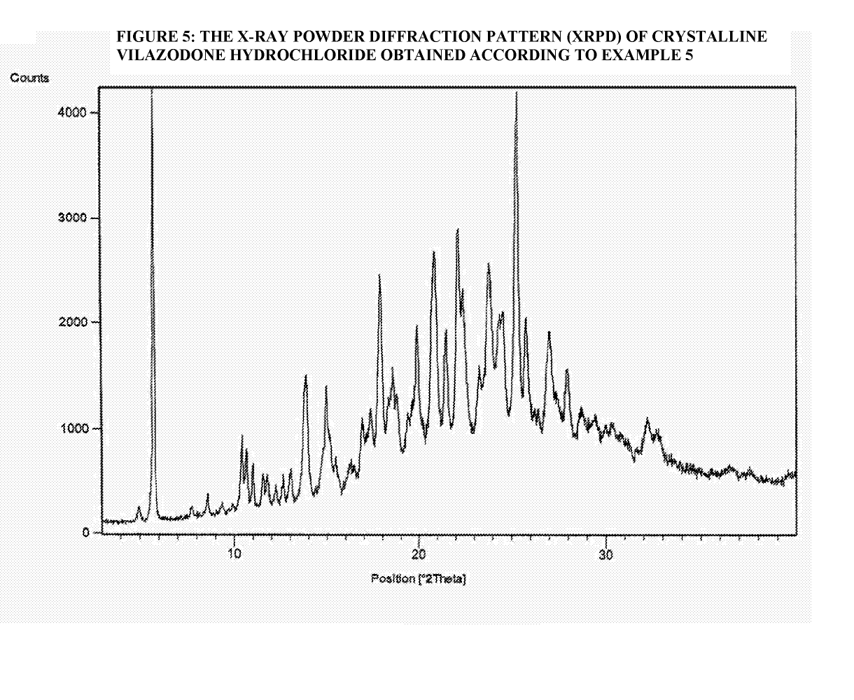

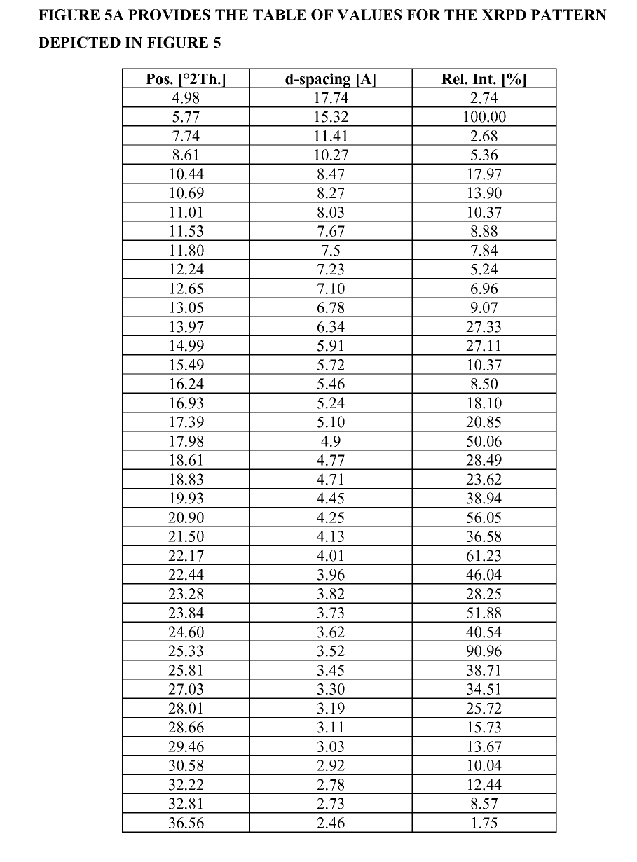

Vilazodone free base (5 g) was added to dichloromethane (25 mL) and ethanol (25 mL) at 20° C. to 30° C. The temperature of the reaction medium was increased to 39° C., and concentrated hydrochloric acid (1.8 mL) was added to the reaction mixture. Deionized water (25 mL) was added to the reaction mixture and the mixture was cooled to 30° C. The reaction mixture was filtered and washed with deionized water (10 mL). The solid obtained was dried under an air dryer at 20° C. to 30° C. for 12 hours and at 85° C. to 90° C. for 10 hours to obtain the title compound having XRPD data as shown in FIG. 5.

Yield: 4.9 g

### Example 6

### Preparation of Vilazodone Hydrochloride

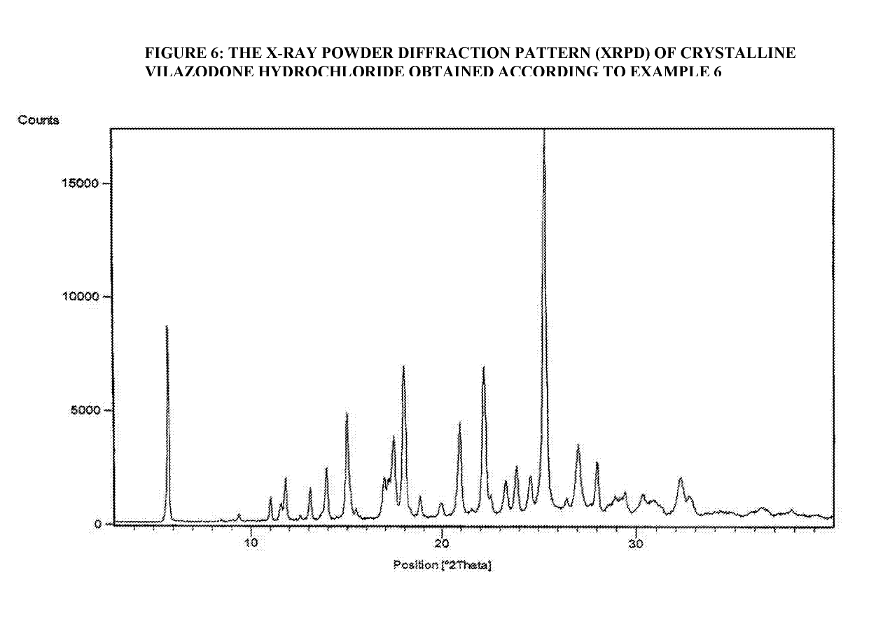

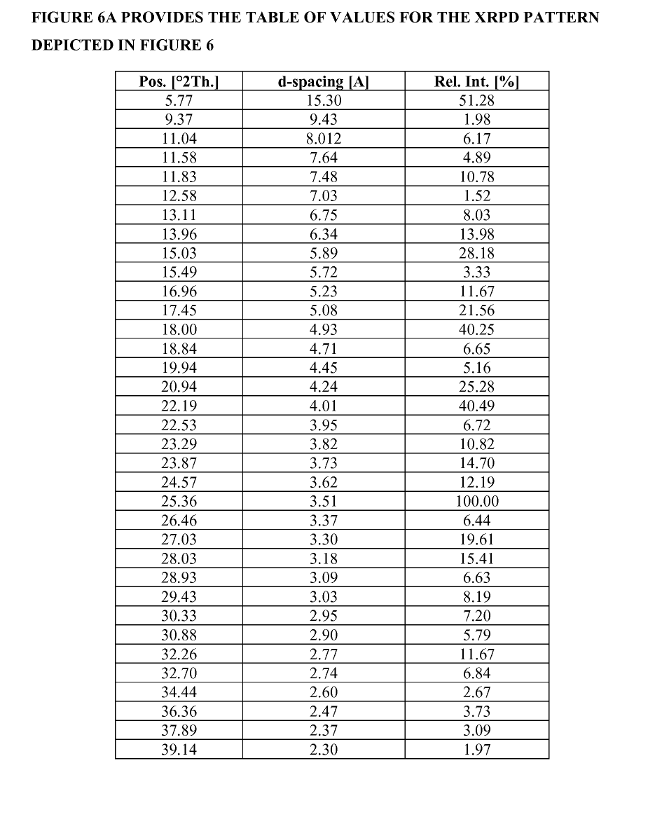

Vilazodone free base (5g) was added to dichloromethane (25 mL) and 2-propanol (25 mL) at 20° C. to 30° C. The temperature of the reaction medium was increased to 40° C., and concentrated hydrochloric acid (1.8 mL) was added to the reaction mixture at 40° C. to 41° C. Deionized water (25 mL) was added to the reaction mixture and the mixture was cooled to 30° C. The reaction mixture was filtered and washed with deionized water (10 mL). The solid obtained was dried under an air dryer at 20° C. to 30° C. for 12 hours and at 85° C. to 90° C. for 10 hours to obtain the title compound having XRPD data as shown in FIG. 6.

Yield: 4.9 g

### Example 7

### Preparation of Vilazodone Hydrochloride

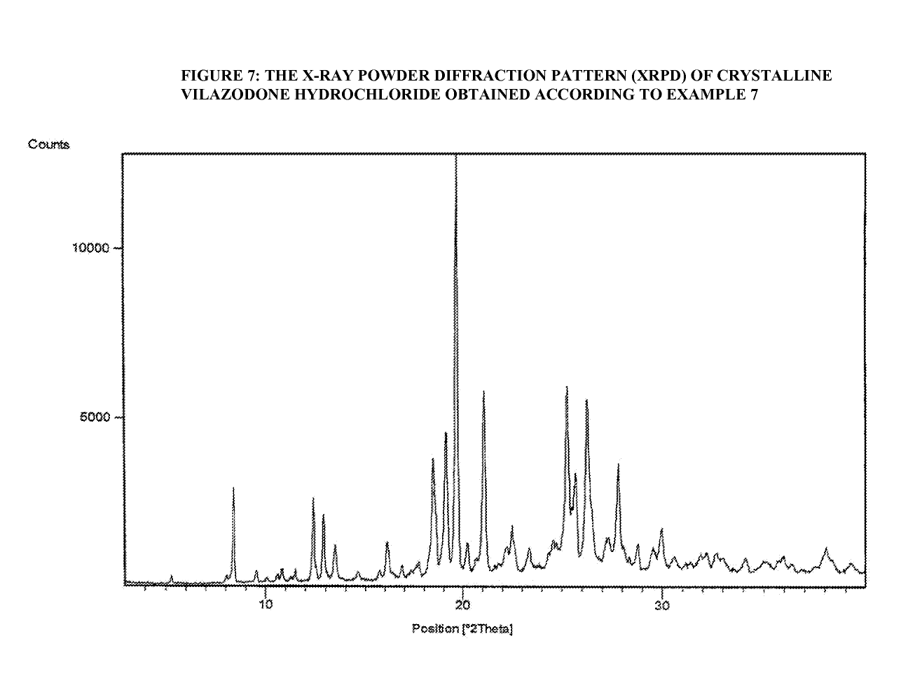

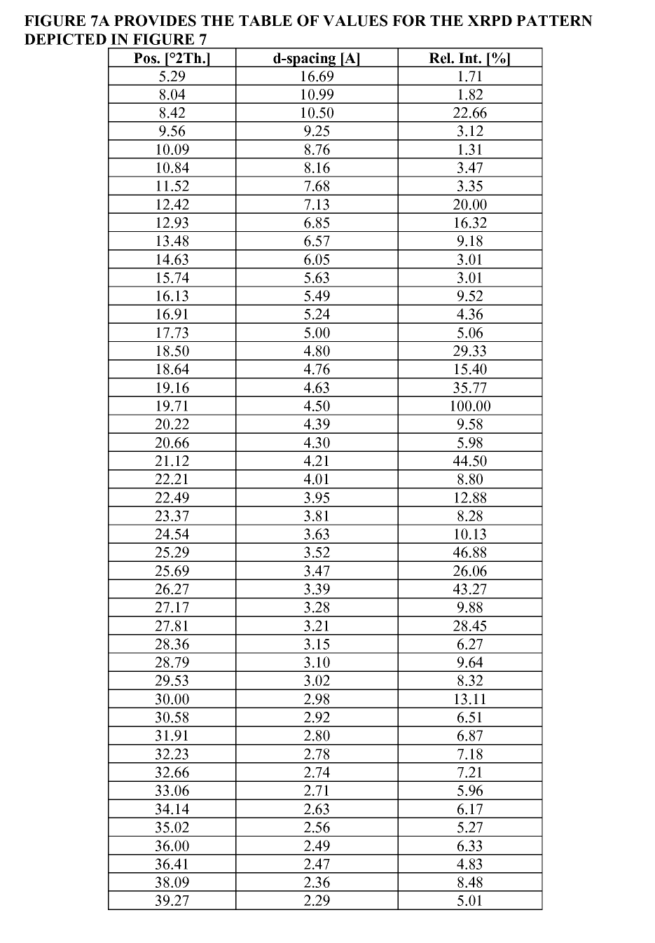

Vilazodone free base (46 g) was added to ethyl acetate (500 mL) and methanol (175 mL) at 20° C. to 30° C. The temperature of the reaction medium was increased to 40° C., and concentrated hydrochloric acid (12.5 mL) was added to the reaction mixture at 40° C. Deionized water (175 mL) was added to the reaction mixture and the mixture was cooled to 30° C. The reaction mixture was filtered and washed with deionized water (100 mL). The solid obtained was dried under an air dryer at 20° C. to 30° C. for 6 hours to obtain the title compound having XRPD data as shown in FIG. 7.

Yield: 24 g

We claim:

1. A process for the preparation of crystalline vilazodone hydrochloride, which comprises:
a) treating vilazodone free base with hydrochloric acid in the presence of water and a solvent selected from the group consisting of alcohol, halogenated hydrocarbon, esters, or a mixture thereof; and
b) isolating crystalline vilazodone hydrochloride from the reaction mixture thereof.

2. The process according to claim 1, wherein the alcohol solvent is methanol, ethanol, 2-propanol, 1-propanol, or butanol.

3. The process according to claim 1, wherein the halogenated hydrocarbon solvent is dichloromethane or chloroform.

4. The process according to claim 1, wherein the ester solvent is ethyl acetate, methyl acetate, or isopropyl acetate.

5. The process according to claim 1, wherein the solvent is ethyl acetate, dichloromethane, 2-propanol, ethanol, methanol, or a mixture thereof.

6. The process according to claim 1, wherein water is added to the reaction mixture before or after the addition of hydrochloric acid.

7. The process according to claim 1, wherein hydrochloric acid is used in solution form or gaseous form.

8. The process according to claim 7, wherein the solution of hydrochloric acid is prepared in 2-propanol.

9. The process according to claim 1, wherein treatment of vilazodone free base with hydrochloric acid is carried out a temperature of about 10° C. to about 100° C.
